(19) United States
(12) Patent Application Publication
(10) Pub. No.: US 2009/0324777 A1
(43) Pub. Date: Dec. 31, 2009

(54) METHOD OF PRODUCING A PALATABILITY ENHANCER THAT CAN ADD HEALTH VALUE TO FOODSTUFFS

(75) Inventors: Haydn Gregg Williams, Rogerstone (GB); Denise Margaret Gallagher, Wateford (IE); Stuart Ian West, Cardiff (GB)

Correspondence Address: DYKEMA GOSSETT PLLC, 10 S. WACKER DR., STE. 2300, CHICAGO, IL, 60606, US

(73) Assignee: BIOCATALYSTS LIMITED, Nantgarw (GB)

(21) Appl. No.: 11/792,214

(22) PCT Filed: Dec. 9, 2005
(86) PCT No.: PCT/GB2005/004735

§ 371 Date: Sep. 11, 2009

(30) Foreign Application Priority Data
Dec. 9, 2004 (GB) 0427077.3

(51) Int. Cl. A23J 3/34; A23L 1/305; A23L 1/227; C12N 9/00

(52) U.S. Cl. 426/41; 426/7; 426/56; 426/52; 426/42; 426/32; 426/47; 426/656; 426/641; 435/183

(57) ABSTRACT

The present invention relates of a method of producing an enzymatic digest of proteinaceous material which can improve the palatability and health value of foodstuffs to which it is added. In particular but not exclusively, the invention involves a method where bioactive compounds are released to increase the health value of the product.

### TECHNICAL FIELD

The present invention relates of a method of producing an enzymatic digest of proteinaceous material which can improve the palatability and health value of foodstuffs to which it is added. In particular but not exclusively, the invention involves a method where bioactive compounds are released to increase the health value of the product.

### BACKGROUND ART

Making food palatable to individuals, whether the individuals are human or animal is very important because if the food is not desirable, it is unlikely to be eaten by the individual which could result in malnutrition. Foods that have good palatability are those which have attractive sensory characteristics, such as smell, texture and flavour as well as being digestible.

Various approaches have been taken to increase the palatability of foods. In U.S. Pat. No. 3,857,968, to G J Haas et al, a process of lypolytic and proteolytic treatment of a mixture of fat and protein is used. This patent describes the production of a complex array of reaction products but no firm reason is given for the increased palatability. However, it is believed that the palatability increase is due to the enhancement of flavour. The product produced is added separately to a final food product, preferably by spraying onto a dried food.

In other patents, for example U.S. Pat. No. 3,617,300 to Borochff et al, the starch content of dog food is altered by enzymatically converting starch to glucose using alpha-amylase and amyloglucosidase, which is added directly to solid dog food. In the case of U.S. Pat. No. 4,391,829 to Spradlin et al, a dog food having farinaceous and meat ingredients is enzymatically modified and coated on the surface of, or admixed with other dog food ingredients.

Problems associated with such prior art palatability enhancers is that some of the conditions used in known techniques to process the food can alter the nutritional content of the foods, and also, some techniques, especially those involving multiple steps can increase production costs of the final food product.

As well as making food palatable, the health content of the food needs to be at an optimum value. Much work has been carried out on isolating materials to be added to food to improve the health value of the food. A particular area of interest in the health additive field is in the area of biologically active food proteins. Bioactive peptides are breakdown products of food proteins by proteases present in the gastrointestinal tract which exhibit a specific biofunction only once they are released from the parent protein. Bioactive peptides are relatively short in length e.g. 2-9 amino acids and possess hydrophobic amino acids, proline, lysine or arginine groups. Recent work has been carried out to isolate bioactive peptides which have been defined as specific protein fragments having a positive impact on the body function or condition which may ultimately affect health (Tranter H S. and Board R G, Journal of Applied Biochemistry 1982: 4:295).

Work has been carried out on different food protein sources to isolate bioactive peptides which may have a desirable physiological and biofunctional activity. Bioactive peptides have been linked with a range of activities including the reduction of hypertension, opioid like activity, antithrombotic activity, the reduction of cholesterol build up, antioxidant activity, antimicrobial activity, immunoregulatory activity and mineral sequestration.

Specific examples of bioactive peptides include antihypertensive peptides known as Angiotensin I converting enzyme (ACE) inhibitors have been obtained from milk, corn and fish protein sources. Peptides with opioid activities have been derived from gluten or casein using Pepsin. The bioactive peptide ovokinin, derived from an enzymatic digest of ovalbumin which is known to have antihypertensive function. There are a number of products containing bioactive peptides, for example Evolus and Ameal S which are fermented milk products. A distinction must be made between bioactive proteins that are present naturally in food, for example growth factors or immunoglobulins that are present in milk and peptides and those that arise as a result of digestion of intact protein sources or from protein hydrolysates as components of food.

Further additives in foods include glucoseaminoglycan which are formed as a result of thermal processing of cartilaginous material e.g. as described in EP 941735 but the process is expensive due to the need to use high temperatures during thermal processing.

Despite the fact that proteinaceous digests are known to increase palatability of foodstuffs, there are no products which have the feature of increased palatability as well as including levels of bioactive peptides which improve the health value of the product. Further the industrial scale production of bioactive peptides is difficult making them expensive to produce by known techniques.

### OBJECTS OF THE INVENTION

The present invention seeks to overcome problems associated with the prior art by providing a method of producing an enzymatic digest for use as a palatability enhancer for foodstuffs that has both increased palatability and improved health value for example reducing hypertension due to ACE inhibitory peptides by an efficient enzymatic method that minimises production costs.

The invention seeks to achieve this by mixing a precursor material which is a source of bioactive compounds with a proteinaceous food base material to form a mixture, the mixture is treated with an enzyme or mixture of enzymes under conditions to form a digest where the proteinaceous food base material is at least partially hydrolysed producing high levels of flavoursome peptides and amino acids and the precursor material is caused to release bioactive compounds, such as bioactive peptides so forming a palatability enhancer with added health benefits.

### DESCRIPTION OF THE INVENTION

According to a first aspect of the invention there is provided a method for producing a palatability enhancer having improved palatability with nutritional and health value, wherein a proteinaceous food base material is mixed with a precursor material which is a source of bioactive compounds to form a mixture, the mixture is treated with one or more enzymes including a proteolytic enzyme at a temperature and pH and for a time period to form a digest where the precursor material is at least partially hydrolysed so as to release bioactive compounds and the proteinaceous base material is at least partially hydrolysed producing flavoursome peptides and amino acids such that when the digest is added to a foodstuff, the palatability, and health value of the foodstuff is enhanced.

Preferably, the method involves thermally treating the digest to inactivate enzymatic activity prior forming or including the palatability enhancer into a final food product.

It is envisaged that the palatability enhancer is combined with a carrier to form a foodstuff or feed.

It is envisaged that the proteinaceous food base material is a meat, a meat based product, or a vegetable protein such as soya or yeast. A combination of one or more of these proteinaceous food base materials may be used.

Preferably, the meat or meat based material is selected from one or a mixture of beef, pork, lamb, poultry, venison, goat, horse, mutton, veal, fish, game, offal, heart or kidneys.

More preferably the meat or meat based material is liver, pancreas or viscera.

Preferably, the meat or meat based material includes vertebrae connective tissue selected from the group consisting of skin, ears, trachea, tendon, nasal septum, arterial walls, skeletal tissue, lungs and mixtures thereof.

It is envisaged that the precursor material is selected from, but not limited to, one or more of casein, milk, whey, egg, albumin, fish, or meat, for example chicken, pork or beef.

Preferably, the precursor material includes vertebrae connective tissue selected from the group consisting of skin, ears, trachea, tendon, nasal septum, arterial walls, skeletal tissue, lungs and mixtures thereof. It is preferred that the connective tissue is bovine tissue as it is of greatest availability in terms of quantity due to the size of the animal that is slaughtered.

The proteinaceous food base material may include other additives depending on what the palatability enhancer that is produced is used for. For human food, in particular additives are included and these can account for up to 30% by weight of the final food product. Often additives such a fruit pastes, fruit juices, sugars, spices and other additives that are used in food processing can be added according to the taste of the target consumer market.

In a preferred embodiment, the mixture includes between 5-40% by uncooked weight of connective tissue and the balance is proteinaceous material.

It is preferred that the mix comprises liver tissue and bovine cartilage. In an alternate embodiment, the mix comprises liver tissue. In a further embodiment, the mix comprises liver, whey protein and bovine cartilage. In another embodiment the mix comprises liver tissue and whey.

Preferably, the proteolytic enzyme is in the form of a preparation including one or more proteases selected from an animal protease, bacterial protease, a fungal protease or a plant protease. In the case of the bacterial protease, this is a neutral or alkaline bacterial protease. The precise concentration at which the proteolytic enzymes are employed is not considered critical as long as the enzymes are present in amounts to cause an enzymatic reaction resulting in increased palatability of the foodstuff and the release of the bioactive peptides and amino acids.

It is preferred that the enzyme dose is equivalent to a 0.1-2% W/W dosage of a common industrial protease such as P144L (200TU) produced by Biocatalysts Ltd. to produce a palatability enhancer with good flavour. More concentrated proteases are available and can be added in smaller volumes based on equivalent protease activity. Alternatively a weaker protease can be added in larger volumes based on equivalent protease activity.

Preferably, the protease enzyme is selected from one or a mixture of endoproteases and exoproteases.

It is envisaged that the protease enzyme is selected from one or mixture of trypsin, papain, bacillolysin, subtilisin, chymotrypsin, bromelain or leucine aminopeptidase.

In a preferred embodiment a mixture of proteases are used to digest a proteinaceous precursor material to produce bioactive peptides.

In a preferred embodiment papain is used in conjunction with trypsin to provide a higher yield of both flavoursome peptides and bioactive peptides.

In a preferred embodiment the proteinaceous precursor material acts as both a source of flavoursome peptides and a source of bioactive peptides.

A preferred embodiment of the invention involves the use of whey as the precursor material to produce bioactive peptides including ACE inhibitors.

A preferred embodiment of the invention involves the use of trypsin to digest whey to produce bioactive peptides.

In a preferred embodiment, the precursor material is a source of Chondroitin Sulphate (CS) and is low in glucosamine sulphate. Chondroitin Sulphate is a glucosaminoglycan produced by cartilage cells called “chondrocytes” and it is essential to cartilage growth, nutrition and repair. Chondroitin sulphate is composed of repeating disaccharide units. The disaccharide unit is composed of D-galactosamine and D-glucuronic acid. The amino sugar in CS may be sulphated in position 4 or 6 resulting in a very high density of negative charge that favours an electrochemical attraction with water, thereby giving cartilage its fluidity and elasticity.

In a preferred embodiment vertebrae connective tissue is used as a source of palatable peptides and chondroitin sulphate.

Preferably, the one or more enzymes used in the reaction include enzymes known to release Chondroitin Sulphate from the precursor material.

Preferably, the protease enzyme is selected from one or a mixture of endoproteases or exoproteases.

It is envisaged that the protease enzyme is selected from one or mixture of trypsin, papain, bacillolysin, subtilisin, chymotrypsin, bromelain or leucine aminopeptidase.

It has been found that the temperatures most suitable for the method range from about 35 degrees C. to approximately 65 degrees C. Temperatures falling within this range are effective for both heating the enzyme reactants prior to adding to the proteinaceous food base material and for maintaining the reaction during the digest phase. Lower temperatures can be used for example from 20 to 35 degrees C. but the reaction is slower and there is also a risk of microbial contamination. Higher temperatures in the range of 65-80 degrees C. can be used but the enzyme may be inactivated.

The pH used during the digest phase can also have an effect on palatability of the foodstuff produced. The pH should be maintained so as to sustain the enzyme conditions to allow the reaction to continue until the bioactive peptides are released and the body of the food is hydrolysed. Typically the pH used is within the range of from about 4 to 9, preferably from 6 to 9 and most preferably from about 6.0 to 8.0, which is neutral to slightly alkaline.

According to a second aspect of the invention there is provided a foodstuff produced by a method according to the first aspect of the invention.

It is envisaged that the foodstuff is an animal foodstuff. However, where acceptable sources of proteinaceous material are used, for example good cuts of lean meat, the foodstuff can be for humans.

It is envisaged that the foodstuff is processed to form a meat product.

Alternatively, the foodstuff may be provided as a tinned product.

It is envisaged that the foodstuff may be combined with other food components to form a processed food product.

According to a further aspect of the invention, there is provided an enzyme or enzyme mix in a carrier solution for use in a method according to a further aspect of the invention.

Preferably the enzyme or enzyme mix is provided in solution. It is envisaged that the solution includes sugars, flavouring or vitamins. However, in an alternative embodiment the enzyme or enzyme mix can be provided as a dried sample.

In a preferred embodiment, the enzyme or enzyme mix comprises enzymes derived from one or more of Pancreas or viscera.

### BEST MODES FOR CARRYING OUT THE INVENTION

The following examples are presented for the purpose of further illustrating and explaining the present invention only. These examples illustrate the efficacy by which palatability enhancers with health benefits may be incorporated into pet foods or animal feed.

### Example 1

A palatability enhancer providing health benefits for animals was prepared from the following formulation, where the composition of substrates used is as set out in the table entitled Composition 1.

### Composition 1: (Substrates Used in Examples 1 and 2)

| Ingredients | Weights |
|---|---|
| Chicken liver | 37.5 wt % |
| Water | 37.5 wt % |
| Bovine cartilage | 25.0 wt % |

The palatability enhancer with health benefits was prepared by blending the chicken liver and water for two minutes at top speed in a food blender. The bovine cartilage material was cut into small pieces and added to the blended chicken liver. A plant derived protease was then added to the formulation at a dose of 0.7% w/w. The enzyme hydrolysis was carried out under agitation for 16 hours at a temperature of about 65° C. The digest was filtered and the concentration of dissolved solids, Chondroitin Sulphate and amino acid content in the filtrate determined. Dissolved solids were measured using a Sartorius moisture analyser, chondroitin sulphate was quantified by the Taylors blue assay (Farndale, R. W. et al, 1986) and the amino acid concentration was determined using the ninhydrin assay method (Moore, S. 1968). The palatability enhancer was heated at 80° C. for 30 minutes to inactivate the protease. The heat treated palatability enhancer was analysed for the presence of ACE inhibitors. The ACE inhibition index of the palatability enhancer was determined by the method described by Murray, B. A. et al (2004).

TABLE 1
Concentration of dissolved solids,
Chondroitin Sulphate, amino acids and ACE inhibitors in
filtered digest.

| | | | | Amino acid | |
|---|---|---|---|---|---|
| | | Dissolved | Chondroitin | content in filtrate | |
| | | solids in | sulphate in | (μmoles of leucine | ACE inhibition |
| | | filtrate | filtrate | equivalents/ml | index* |
| Substrate | Enzyme(s) | (mg/g) | (mg/ml) | filtrate) | (%) |
| Example 1 | Papain | 132.55 | 13.67 | 307 | 46.56 |

*Inhibition index is a measure of the inhibitory potency of a known test inhibitor substance, in this case 50 μl test inhibitor substance (10 mg freeze-dried powder/ml in borate buffer), in comparison to that ACE activity obtained in the absence of inhibitor.

### Example 2

The substrate and first enzyme hydrolysis stage were carried out as described in Example 1 using composition 1 as described above. On completion of the first enzymatic stage a sufficient amount of 2M sodium hydroxide was added to adjust the pH of the protein hydrolysate to about 8.0. A second protease, trypsin, was then added at a dosage of 0.15% w/w and thoroughly dispersed by mixing. The second enzymatic hydrolysis step was carried out at a temperature of 50° C. for 5 hours. The digest was filtered and the concentration of dissolved solids, chondroitin sulphate and amino acid content in the filtrate determined. The palatability enhancer was heated at 80° C. for 30 minutes to inactivate the protease. The heat treated palatability enhancer was analysed for the presence of ACE inhibitors.

TABLE 2
Concentration of dissolved solids,
Chondroitin Sulphate, amino acids and ACE inhibitors in
filtered digest.

| | | | | Amino acid content | |
|---|---|---|---|---|---|
| | | Dissolved | Chondroitin | in filtrate (μmoles of | |
| | | solids in | sulphate in | leucine | ACE inhibition |
| | | filtrate | filtrate | equivalents/ml | index |
| Substrate | Enzyme(s) | (mg/g) | (mg/ml) | filtrate) | (%) |
| Example 2 | Papain & | 120.40 | 9.75 | 352 | 49.39 |
| | Trypsin | | | | |

Tables 1 & 2 illustrate the effect of the enzyme papain and a mixture of the enzymes papain and trysin on the substrate mixture described in Example 1. In both samples there is release of Chondroitin Sulphate as well as amino acids/bioactive peptides.

### Example 3

A palatability enhancer providing health benefits for animals was prepared from the following formulation:

### Composition 2 (Substrates Used for Example 3).

| Ingredients | Weights |
|---|---|
| Chicken liver | 37.5 wt % |
| Water | 37.5 wt % |
| Whey protein | 25.0 wt % |

The palatability enhancer with health benefits was prepared by blending the chicken liver and water for two minutes at top speed in a food blender. The whey protein was added and mixed well to ensure that the whey protein was dispersed thoroughly within the substrate mixture. A plant derived protease was then added to the formulation at a dose of 0.7% w/w. The enzyme hydrolysis was carried out under agitation for 16 hours at a temperature of about 65° C. The digest was filtered and the concentration of dissolved solids, and amino acid content in the filtrate determined. The palatability enhancer was heated at 80° C. for 30 minutes to inactivate the protease. The heat treated palatability enhancer was analysed for the presence of ACE inhibitors.

TABLE 3
Concentration of dissolved solids, amino acids
and ACE inhibitors in filtered digest.

| | | Dissolved | Amino acid content in | ACE |
|---|---|---|---|---|
| | | solids in | filtrate (μmoles of | inhibition |
| | | filtrate | leucine equivalents/ml | index |
| Substrate | Enzyme(s) | (mg/g) | filtrate) | (%) |
| Example 3 | Papain | 272.70 | 1456 | 67.96 |

### Example 4

A palatability enhancer providing health benefits for animals was prepared from the following formulation as shown by the following “Composition 3”.

### Composition 3 (Substrates Used for Example 4).

| Ingredients | Weights |
|---|---|
| Chicken liver | 45.0 wt % |
| Water | 45.0 wt % |
| Whey protein | 10.0 wt % |

The palatability enhancer with health benefits was prepared by blending the chicken liver and water for two minutes at top speed in a food blender. The whey protein was added and mixed well to ensure that the whey protein was dispersed thoroughly within the substrate mixture. The pH of the substrate was adjusted to about pH 8.0 using 2M sodium hydroxide. An animal derived protease, trypsin, was added to the formulation at a dose of 0.15% w/w and thoroughly dispersed. The enzyme hydrolysis was carried out under agitation for 16 hours at a temperature of about 50° C. The digest was filtered and the concentration of dissolved solids, and amino acid content in the filtrate determined. The palatability enhancer was heated at 80° C. for 30 minutes to inactivate the protease. The heat treated palatability enhancer was analysed for the presence of ACE inhibitors.

TABLE 4
Concentration of dissolved solids, amino acids
and ACE inhibitors in filtered digest.

| | | Dissolved | Amino acid content in | ACE |
|---|---|---|---|---|
| | | solids in | filtrate (μmoles of | inhibition |
| | | filtrate | leucine equivalents/ml | index |
| Substrate | Enzyme(s) | (mg/g) | filtrate) | (%) |
| Example 4 | Trypsin | 148.30 | 858 | 24.05 |

Table 4 shows the relative effect of trypsin hydrolysis of chicken liver material and whey protein. As there is no cartilage material, no Chondroitin Sulphate is released.

### Example 5

A palatability enhancer providing health benefits for animals was prepared from the following formulation:

### Composition 3 (Used in Examples 5 & 6).

| Ingredients | Weights |
|---|---|
| Chicken liver | 32.5 wt % |
| Water | 32.5 wt % |
| Whey protein | 10.0 wt % |
| Bovine cartilage | 25.0 wt % |

The palatability enhancer with health benefits was prepared by blending the chicken liver and water for two minutes at top speed in a food blender. The bovine cartilage material was cut into small pieces and added to the blended chicken liver. The whey protein was added and mixed well to ensure that the whey protein was dispersed thoroughly within the substrate mixture. A plant derived protease was then added to the formulation at a dose of 0.7% w/w. The enzyme hydrolysis was carried out under agitation for 16 hours at a temperature of about 65° C. The digest was filtered and the concentration of dissolved solids, Chondroitin Sulphate and amino acid content in the filtrate determined. The palatability enhancer was heated at 80° C. for 30 minutes to inactivate the protease. The heat treated palatability enhancer was analysed for the presence of ACE inhibitors.

TABLE 5
Concentration of dissolved solids,
Chondroitin Sulphate, amino acids and ACE inhibitors in
filtered digest.

| | | | | Amino acid | |
|---|---|---|---|---|---|
| | | | | content in | |
| | | | Chondroitin | filtrate (μmoles | |
| | | Dissolved | sulphate in | of leucine | ACE inhibition |
| | | solids in filtrate | filtrate | equivalents/ml | index |
| Substrate | Enzyme(s) | (mg/g) | (mg/ml) | filtrate) | (%) |
| Example 5 | Papain | 192.80 | 10.59 | 1260 | 59.74 |

### Example 6

According to this example the substrate and first enzyme hydrolysis stage were as described in Example 5. On completion of the first enzymatic stage a sufficient amount of 2M sodium hydroxide was added to adjust the pH of the protein hydrolysate to about 8.0. A second protease, trypsin, was then added at a dosage of 0.15% w/w and thoroughly dispersed. The second enzymatic hydrolysis step was carried out at a temperature of 50° C. for 5 hours. The digest was filtered and the concentration of dissolved solids, Chondroitin Sulphate and amino acid content in the filtrate determined. The palatability enhancer was heated at 80° C. for 30 minutes to inactivate the protease. The heat treated palatability enhancer was analysed for the presence of ACE inhibitors.

TABLE 6
Concentration of dissolved solids,
Chondroitin Sulphate, amino acids and ACE inhibitors in
filtered digest.

| | | | | Amino acid content | |
|---|---|---|---|---|---|
| | | Dissolved | Chondroitin | in filtrate (μmoles of | |
| | | solids in | sulphate in | leucine | ACE inhibition |
| | | filtrate | filtrate | equivalents/ml | index |
| Substrate | Enzyme(s) | (mg/g) | (mg/ml) | filtrate) | (%) |
| Example 6 | Papain & | 180.50 | 13.98 | 732 | 70.08 |
| | trypsin | | | | |

Tables 5 & 6 shows the production of both Chondroitin Sulphate and amino acids, with Example 5 using 0.7% of a plant derived protease and a further 0.15% w/w of trypsin for Example 6.

### Example 7

A palatability enhancer providing health benefits for animals was prepared from the following formulation:

### Composition 4 (Substrates Used in Example 7).

| Ingredients | Weights |
|---|---|
| Chicken liver | 30.0 wt % |
| Water | 30.0 wt % |
| Bovine cartilage | 40.0 wt % |

The palatability enhancer with health benefits was prepared by blending the chicken liver and water for two minutes at top speed in a food blender. The bovine cartilage material was cut into small pieces and added to the blended chicken liver. A plant derived protease was then added to the formulation at a dose of 0.7% w/w. The enzyme hydrolysis was carried out under agitation for 16 hours at a temperature of about 65° C. The digest was filtered and the concentration of dissolved solids, Chondroitin Sulphate and amino acid content in the filtrate determined. The palatability enhancer was heated at 80° C. for 30 minutes to inactivate the protease. The heat treated palatability enhancer was analysed for the presence of ACE inhibitors.

TABLE 7
Concentration of dissolved solids,
Chondroitin Sulphate, amino acids and ACE inhibitors in
filtered digest.

| | | | | Amino acid content | |
|---|---|---|---|---|---|
| | | Dissolved | | in filtrate (μmoles of | |
| | | solids in | Chondroitin | leucine | ACE inhibition |
| | | filtrate | sulphate in | equivalents/ml | index |
| Substrate | Enzyme(s) | (mg/g) | filtrate (mg/ml) | filtrate) | (%) |
| Example 7 | Papain | 131.05 | 15.89 | 507.09 | 48.02 |

Table 7 shows increased levels of Chondroitin Sulphate, the higher the level of cartilage material in a sample.

Modification and variations of the present invention will become apparent to those skilled in the art and it is intended that all such modifications will be included within the scope of the present invention. The invention is intended to cover not only single embodiments of the invention but also combinations of those embodiments.

1. A method for producing a palatability enhancer for a foodstuff wherein a proteinaceous food base material is mixed with a precursor material which is a source of bioactive compounds to form a mixture, the mixture is treated with one or more enzymes including a proteolytic enzyme to form a digest where the precursor material is at least partially hydrolysed to release bioactive compounds and the proteinaceous base material is at least partially hydrolysed to produce peptides and amino acids such that when the digest is added to a foodstuff, the digest provides enhanced palatability and health value to the foodstuff by way of the peptides and amino acids and bioactive components.

2. The method according to claim 1, wherein the method is carried out at a temperature from 35 degrees C. to 65 degrees C.

3. The method according to claims 1 or 2 wherein the method is carried out at a pH selected from one of the following, 4 to 9, 6 to 9 or 6 to 8.

4. The method according to claim 3, wherein the proteinaceous food base material is selected from one or more of a meat, a meat based product, or a product including vegetable protein.

5. The method according to claim 4, wherein the meat or meat based material is selected from one or more of beef, pork, lamb, poultry, venison, goat, horse, mutton, veal, fish, game, offal, heart, liver, pancreas, viscera or kidneys and mixtures thereof.

6. The method according to claim 4, wherein the meat or meat based material includes connective tissue selected from the group consisting of skin, ears, trachea, tendon, nasal septum, arterial walls, skeletal tissue, lungs and mixtures thereof.

7. The method according to claim 1, wherein the precursor material is selected from one or more of casein, milk, whey, egg, albumin, fish, meat or combinations thereof.

8. The method according to claim 7, wherein the precursor material includes connective tissue selected from the group consisting of skin, eats, trachea, tendon, nasal septum, arterial walls, skeletal tissue, lungs and mixtures thereof.

9. The method according to claim 8, wherein the connective tissue is bovine tissue.

10. The method according to claim 1, wherein the proteinaceous food base material includes additives.

11. The method according to claim 10, wherein the additives are present at an amount of up to 30% by weight of the final food product.

12. The method according to claims 10 or 11, wherein the additives are selected from one or more of fruit pastes, fruit juices, sugars or spices.

13. The method according to claim 1, wherein the proteinaceous part of the mixture includes between 5-40% by uncooked weight of connective tissue with the balance being proteinaceous material.

14. The method according to claim 1, wherein the mixture comprises either:
a) liver tissue and bovine cartilage; or
b) liver tissue; or
c) liver, whey protein and bovine cartilage; or
d) liver tissue and whey.

15. The method according to claim 1, wherein the proteolytic enzyme includes one or more proteases selected from an animal protease, bacterial protease, a fungal protease or a plant protease.

16. The method according to claim 15, wherein when a bacterial protease is used said bacterial protease is a neutral or alkaline bacterial protease.

17. The method according to claims 15 or 16, wherein the protease is selected from, or is a mixture of endoproteases and exoproteases.

18. The method according to claim 17, wherein the protease enzyme is selected from one or more of trypsin, papain, bacillolysin, subtilisin, chymotrypsin, bromelain or leucine aminopeptidase or a mixture thereof.

19. The method according to claim 18, wherein when papain is used it is used in conjunction with trypsin.

20. The method according to claim 15, wherein the proteolytic enzyme dose is equivalent to a 0.1-2% W/W dosage.

21. The method according to claim 1, wherein the bioactive peptides include Angiotensin converting enzyme (ACE) inhibitors.

22. The method according to claim 1, wherein the precursor material is a source of Chondroitin Sulphate (CS).

23. The method according to claim 22, wherein the precursor material is low in glucosamine sulphate.

24. The method according to claim 1, wherein the palatability enhancer is combined with a carrier.

25. The method according to claim 1, wherein the digest is thermally treated to inactivate enzymatic activity prior to including the palatability enhancer into a final food product.

26. A digest produced by a method according to claim 1.

27. The digest according to claim 26, wherein the health value that it provides, when added to a food is one or more of hypertension reduction, opioid type activity, antithrombotic activity, cholesterol reduction, antioxidant activity, antimicrobial activity, immunoregulatory activity or mineral sequestration.

28. An enzyme or enzyme mix in a carrier solution for use in a method according to claim 1 to produce a digest.

29. The enzyme mix according to claim 28, wherein the solution includes sugars, flavouring or vitamins.

30. The enzyme mix according to claims 28 or 29, wherein the solution is dried to form a dried sample.

31. The enzyme mix according to claim 28 wherein the enzyme or enzyme mix comprises enzymes derived from one or more of pancreas of viscera.

32. A foodstuff to which a digest has been added according to claims 26 or 27.

33. The foodstuff according to claim 32 wherein the foodstuff is a meat product.

34. The foodstuff according to claim 32, wherein the foodstuff is combined with other food components to form a final processed food product.
